(19) United States
(12) Patent Application Publication
(10) Pub. No.: US 2025/0153153 A1
(43) Pub. Date: May 15, 2025

(54) CATALYST FOR CATALYTIC CRACKING OF MIXED RAW MATERIAL OF DIESEL AND METHANOL AND PREPARATION METHOD THEREFOR

(71) Applicant: Korea Research Institute of Chemical Technology, Daejeon (KR)

(72) Inventors: Na Young Kang, Daejeon (KR); Yong Ki Park, Daejeon (KR); Do Kyoung Kim, Daejeon (KR); Ji Ho Shin, Daejeon (KR); Tae Young Jung, Daejeon (KR); Hong Jin Lee, Daejeon (KR); Won Kyung Moon, Daejeon (KR)

(21) Appl. No.: 19/021,173

(22) Filed: Jan. 15, 2025

Related U.S. Application Data
(63) Continuation of application No. PCT/KR2023/010111, filed on Jul. 14, 2023.

(30) Foreign Application Priority Data
Jul. 15, 2022 (KR) 10-2022-0087768

(51) Int. Cl. B01J 29/40; B01J 37/04; B01J 37/08; C10G 11/05

(52) U.S. Cl. CPC B01J 29/40; B01J 37/04; B01J 37/08; C10G 11/05; C10G 2300/1055; C10G 2300/4006; C10G 2300/4012; C10G 2300/70; C10G 2400/20

(57) ABSTRACT

The present invention relates to a catalyst and a preparation method therefor, wherein the catalyst can convert diesel, which is increasingly likely to be unused, to a basic chemical raw material, and induce a thermal polymerization reaction by combining a strong endothermic reaction of diesel with an olefin reaction of methanol, which is a type of exothermic reaction, to minimize the amount of energy used in a catalytic cracking reaction of diesel. An aspect of the present invention provides a catalyst for use in a reaction for preparing a hard olefin by catalytic cracking of a mixture of diesel and methanol, and is technically characterized in that the catalyst comprises: porous zeolite with pores; and phosphorus supported inside the pores of the porous zeolite and/or on the surface of the porous zeolite, and the catalyst has a retention time of isobutane gas in the pores of 0.400-1.240 min/g.

### TECHNICAL FIELD

The present invention relates to a catalyst which produces light olefins by catalytic cracking of a mixed raw material of diesel and methanol, a method for preparing the same, and a use thereof.

### BACKGROUND ART

Diesel is a mixture of hydrocarbons having a boiling point of 250 to 350° C. and is mainly used as a fuel for passenger cars, buses, trucks, small and medium-sized ships, and large passenger planes. Recently, as a demand for electric vehicles rapidly increases due to global warming which is a global environment issue, a demand for heavier fractions than naphtha, such as diesel and kerosine, is expected to stagnate or decline, by change in the transport dynamometer and development of alternative renewable energy related thereto. Accordingly, development of technology capable of selectively producing light olefins which are basic chemical raw material, by a decomposition reaction of a diesel fraction, of which the unused amount in an essential oil refinery is expected to increase in the future, is demanded.

Since there has been no commercial case of catalytic technology for converting a diesel fraction into a basic chemical raw material yet, and a thermal decomposition reaction for inducing light materials by a decomposition reaction itself is a strong endothermic reaction, a reaction at a high temperature of 500° C. or higher is needed, but when a solid acid catalyst is used, energy usage to activate resolution may be decreased, and thus, the decomposition technology using the solid acid catalyst may be selected as a good candidate technology.

Among the similar catalyst technologies, in “Steam catalytic cracking of n-dodecane over Ni and Ni/Co Bimetallic catalyst supported on Hierarchical BEA zeolite” (Energy Fuels 2017, vol 31, p 5482-5490) which reports a catalytic decomposition reaction technology based on BEA zeolite using n-dodecane as a model compound of heavy oil, a benefit of a catalytic technology in which micropores in zeolite is converted into a 3D connectable hierarchical structure by dissolving silica in zeolite with strong alkali to support a metal is described. It was reported that when BEA having a hierarchical structure in which cobalt is supported is used, ethylene, propylene, and butene may be produced with a total yield of up to 28.6 wt % at a reaction temperature of 400° C. However, the prior art designed a catalyst only considering the decomposition properties of paraffin which accounts for only about 20% of a diesel fraction and has no technical consideration for targeting hydrocarbons corresponding to naphthene and aromatics.

In addition, in “Cracking performance of gasoline and diesel fractions from catalytic pyrolysis of heavy gas oil derived from Canadian synthetic crude oil” (Energy fuels 2011, vol 25, p3382-3388) which reports a decomposition reaction technology by a catalytic pyrolysis reaction using heavy gas oil, it is described that ethylene and propylene were able to be produced with a total yield up to about 10 wt % at a reaction temperature of 700° C. from a molding catalyst including ZSM-5 zeolite. The prior art has no description for a specific catalyst structure for improving an olefin yield, even though a decomposition reaction was induced at a high temperature than a conventional naphtha decomposition temperature, and specifies only a possibility of converting heavy gas oil into a basic chemical raw material without technical consideration for improving catalytic properties.

A naphtha catalytic decomposition process (K-COT™) which was commercialized in 2017 still consumes much energy even though a reaction temperature was lowered by 150° C. or higher as compared with a conventional thermal decomposition process and has a limitation in producing an excessive amount of methane, and thus, in order to improve the situation, recently, technologies of combining the endothermic reaction characteristics of a naphtha cracking reaction and the exothermic reaction characteristics of a methanol conversion reaction to achieve heat neutralization to minimize energy usage have been developed. In this regard, Korean Patent Registration No. 10-1803406 reported a technology promoting process efficiency by co-feeding a raw material such as methanol as well as naphtha as a reaction raw material in a naphtha catalytic cracking reaction. It is described that the catalytic cracking reaction process according to the invention may promote heat neutralization by using a circulating fluidized bed reactor and changing input positions of naphtha and methanol which are put into a reactor to decompose naphtha and methanol simultaneously, energy usage may be minimized and production of light saturated hydrocarbons such as methane, ethane, and propane is suppressed to improve a light olefin yield. Naphtha is a mixture of hydrocarbons having a boiling point in a range of 30 to 200° C., and in the patent, a ZSM-5 based molding catalyst was used in order to derive naphtha into an olefin, but a catalyst which may decompose only a fraction in a range of C₅-C₈ having a smaller molecular weight than diesel was used.

Korean Patent Registration No. 10-1550202 discloses a catalyst for preparing light olefins and/or aromatic hydrocarbons by a catalytic cracking reaction of a mixture including naphtha and methanol, which includes 25 to 80 wt % of a ZSM-5 molecular sieve, 15 to 70 wt % of a binder, and 2.2 to 6.0 wt % of lanthanum and 1.0 to 2.8 wt % of phosphorus which are loaded on the ZSM-5 molecular sieve. However, the catalytic composition of the patent is similar to the composition of the conventional naphtha decomposition catalyst, and there is a limitation in applying the conventional catalyst as it is in order to react diesel and methanol simultaneously.

Thus, the present inventors found that the selectivity of light olefins may be increased by controlling an acid site of a specific zeolite to adjust the mass transfer rate of reactants in zeolite, in order to decompose diesel and methanol formed of various and complicated structures in a range of C₁₀-C₂₀ simultaneously, and thus, completed the present invention.

### DISCLOSURE OF INVENTION

### Technical Problem

In the present invention, in order to prepare light olefins by catalytic cracking of a mixed raw material of diesel and methanol, phosphorus (P) was introduced to a specific zeolite, thereby controlling an acid site to adjust a diffusion rate of reactants in the zeolite, and thus, an object of the present invention is to provide a catalyst which may maximize productivity of olefins and a method for preparing the same.

### Technical Solution

In one general aspect, a catalyst used in a reaction for preparing light olefins by catalytic cracking of a mixture including diesel and methanol includes: a porous zeolite having pores; and phosphorus supported inside the pores of the zeolite and/or on a surface of the zeolite, wherein the catalyst has a residence time of an isobutane gas in the pores of 0.400 to 1.240 min/g.

The catalyst may have the residence time of the isobutane gas in the pores of 0.432 to 1.064 min/g.

The catalyst may satisfy the following Equation 1:

$$0.1 \leq P / A 1 \leq 2.5 \qquad [ Equation 1 ]$$

wherein P is a content (mol) of phosphorus supported on the zeolite, and Al is a content (mol) of aluminum in the zeolite.

A P/Al mole ratio may be 0.4 to 0.9.

The phosphorus-supported zeolite may have a total acid site (Aa) of 0.200 to 0.620 mmol/g.

The phosphorus-supported zeolite may have a surface acid site (As) of 0.050 to 0.150 mmol/g.

The phosphorus-supported zeolite may satisfy the following Equation 2:

$$0.2 \leq As / Aa \leq 0.4 \qquad [ Equation 2 ]$$

wherein As is a surface acid site (mmol/g) of the phosphorus-supported zeolite, and Aa is a total acid site (mmol/g) of the phosphorus-supported zeolite.

The zeolite may have a Si/Al mole ratio of 200 or less and include at least one selected from the group consisting of ZSM-11, ZSM-5, Beta, Mordenite, chabazite, or Ferrierite.

The zeolite may have a Si/Al mole ratio of 8 to 30 and may be ZSM-11 or ZSM-5.

The phosphorus-supported zeolite may satisfy the following Equation 3:

$$2. \leq At / Aa ( min / m mol ) \leq 4. \qquad [ Equation 3 ]$$

wherein At is the residence time (min/g) of the isobutane gas in the pores of the catalyst, and Aa is the total acid site (mmol/g) of the phosphorus-supported zeolite.

In another general aspect, a method for preparing the catalyst includes: (a) mixing a porous zeolite having pores and a phosphorus compound at contents corresponding to a P/Al mole ratio of 0.1 to 2.5 in a solvent; (b) drying the mixture at 100 to 150° C. for 5 to 20 hours; and (c) firing a product of the drying at 400 to 600° C. for 2 to 10 hours.

The phosphorus compound may include a phosphoric acid, ammonium phosphate, and/or alkyl phosphate.

The porous zeolite having pores may have a Si/Al mole ratio of 200 or less and include at least one selected from the group consisting of ZSM-11, ZSM-5, Beta, Mordenite, chabazite, or Ferrierite.

The drying (b) may further include evaporating the solvent in the mixture of the mixing (a) in an evaporator at room temperature.

In still another general aspect, a method for preparing light olefins includes catalytically cracking reactants including diesel, methanol, or a mixed raw material thereof in the presence of the catalyst.

The mixed raw material may have a weight ratio of diesel to methanol of 0.1 to 5.

The catalytic cracking may be performed under reaction conditions of a reaction temperature of 600 to 700° C., a reaction pressure of 0.1 to 2 bar, and a weight ratio of the catalyst to the reactants of 2 to 20.

The reactants and a product of the catalytic cracking may have an olefin weight ratio of 1:5 to 1:100.

### Advantageous Effects

The present invention relates to a catalyst which may derive a heat neutralization reaction by combining a strong endothermic reaction of diesel and an olefin reaction of methanol which is an exothermic reaction in order to convert diesel which is increasingly likely to be unused into a basic chemical raw material and minimize energy usage during the catalytic decomposition reaction of diesel, and a method for preparing the same, and is expected to be commercially applied for production of chemical raw materials in the refining and petrochemical industries aiming at carbon neutrality in the future by controlling a specific zeolite acid site in the catalytic cracking reaction of the mixed raw material of diesel and methanol.

### BRIEF DESCRIPTION OF DRAWINGS

FIG. 1 is a graph showing a temperature change of a catalyst layer in a reactor during catalytic cracking of mixed reactants of diesel and methanol and shows results of using the catalyst prepared in Example 1.

### BEST MODE FOR CARRYING OUT THE INVENTION

Unless otherwise defined herein, all terms used herein (including technical and scientific terms) may have the meaning that is commonly understood by those skilled in the art to which the present invention pertains. Throughout the present specification, unless explicitly described to the contrary, “comprising” any elements will be understood to imply further inclusion of other elements rather than the exclusion of any other elements. In addition, unless otherwise particularly mentioned, a singular form includes a plural form herein.

In the present specification, “zeolite” is used with the same meaning as the “porous zeolite having pores”.

An exemplary embodiment of the present invention provides a catalyst used in a reaction

for preparing light olefins by catalytic cracking of a mixture including diesel and methanol. The catalyst is a catalyst including: a porous zeolite having pores; and phosphorus supported inside the pores of the zeolite and/or on a surface of the zeolite, wherein the catalyst has a residence time of an isobutane gas in the pores of 0.400 to 1.240 min/g.

The catalyst of the present invention is a catalyst for a catalytic cracking reaction which catalytically cracks reactants including diesel and methanol to produce light olefins. A solid acid catalyst such as zeolite which is applied in the present invention forms a Bronsted and/or Lewis acid site on a solid surface, and may be used in various process such as catalytic cracking, isomerization, and alkylation of a hydrocarbon reactant in petrochemical industry. Since silicon (Si) and aluminum (Al) of the zeolite are bonded to each other using oxygen as a bridge, an acid site may be produced therefrom. According to the present invention, the porous zeolite catalyst having phosphorus (P) supported inside the pores and on the surface may have improved selectivity of light olefins by decreasing gas by-products of alkanes produced by a diesel decomposition reaction and a methanol conversion reaction, and may have further enhanced light olefin selectivity by adjusting a pore opening size and the acid site in zeolite. It is analyzed that the effect is related to the residence time of the isobutane gas in the pores of the catalyst and the acid site in the catalyst described later.

Meanwhile, the content of phosphorus in the present invention may refer to an amount of phosphorus oxide (P₂O₅) calculated according to the stoichiometric amount based on the case in which a precursor supported on zeolite during preparation of the catalyst is all converted into phosphorus oxide (P₂O₅).

The zeolite of the present invention may have a Si/Al mole ratio of 200 or less, preferably 8 to 30. When aluminum in zeolite is excessively increased, thermal and hydrothermal stability may be lowered, and on the contrary, when silicon is increased, an amount of acid site produced by aluminum is decreased to decrease catalytic activity, and also, economic feasibility is not good in molecular sieve synthesis. Meanwhile, the zeolite may have a FER, MFI, MEL, BEA, CHA, or MOR structure, and preferably zeolite having a MEL structure may be more preferred since it has a high light olefin selectivity. Specifically, the zeolite of the present invention may include at least one selected from the group consisting of Y, SAPO-11, SAPO-34, ZSM-11, ZSM-5, Beta, Mordenite, chabazite, or Ferrierite, and may be preferably ZSM-11 or ZSM-5 and most preferably ZSM-11. According to this, light olefins may be prepared with high selectivity during simultaneous catalytic cracking of diesel and methanol, and simultaneously, a produced amount of by-products such as carbon monoxide and methane may be minimized.

The catalyst of the present invention may satisfy a ratio of the content of phosphorus to the content of aluminum (moles of phosphorus/moles of aluminum) in zeolite represented by the following Equation 1:

$$0.1 \leq P / A 1 \leq 2.5 \qquad [ Equation 1 ]$$

wherein P is a content (mol) of phosphorus supported on the zeolite, and Al is a content (mol) of aluminum in the zeolite.

The P/Al mole ratio may be specifically 0.1 or more, 0.2 or more, 0.3 or more, or 0.4 or more and 2.5 or less, 2.0 or less, 1.5 or less, less than 1.0, or 0.9 or less, and preferably 0.4 to 0.9. When the P/Al mole ratio of the phosphorus-supported zeolite catalyst is within the range described above, it may be designed as a catalyst appropriate for catalytic cracking of a mixture of diesel and methanol. In the examples also, as described above, the light olefins may be prepared with a high yield during catalytic cracking of the mixture including diesel and methanol using the present catalyst, and it is analyzed that the results are related to the total acid site amount, the surface acid site amount, and the residence time of an isobutane gas in the pores of the catalyst described later. Meanwhile, when the content of supported phosphorus is too small, an effect of increasing hydrothermal stability by phosphorus is decreased and the total number of acid sites neutralized by phosphorus is small, so that the initial acid strength of the catalyst is excessively high to lower the light olefin yield by catalytic cracking of the mixed raw material. When the content of supported phosphorus is too high, the total acid site may be excessively neutralized by phosphorus, and also the pores are blocked to greatly decrease the specific surface area of the catalyst, thereby decreasing catalytic activity.

As a result, selectivity of the desired olefin may be increased by controlling the amount of acid site of the material in contact during catalytic decomposition to be appropriate for the corresponding raw material. When heavy diesel is used as a catalytic cracking reactant as in the present invention, it was analyzed that i) generally, the longer the hydrocarbon is, the easier it is to decompose it, and ii) when the amount of acid site of the catalyst is too large or the acid strength is high, meta-stable olefins participate in the reaction again as a reaction intermediate and is easily converted into methane or aromatic, and it was found that adjusting the amount of acid site in the zeolite catalyst may be the most important issue of development of a catalyst material for preparing light olefins of heavy fractions such as diesel.

The total acid site of the phosphorus-supported zeolite of the present invention may be 0.200 to 0.620 mmol/g. Specifically, the total acid site may be 0.200 mmol/g or more or 0.220 mmol/g or more and 0.620 mmol/g or less, 0.600 mmol/g or less, 0.500 mmol/g or less, 0.400 mmol/g or less, 0.350 mmol/g or less, or 0.300 mmol/g or less and preferably, may be 0.208 to 0.320 mmol/g. By adjusting the total acid site of zeolite by supporting phosphorus inside the pores and/or on the surface, production of by-products such as methane, ethane, and propane gas may be decreased and the selectivity of light olefins may be improved.

Meanwhile, the surface acid site of the phosphorus-supported zeolite of the present invention may be 0.050 to 0.150 mmol/g. Specifically, the surface acid site may be 0.060 to 0.080.

In the conventional technology, production of coke on the surface is suppressed by further decreasing the acid site positioned on the surface of the total acid site, so that catalyst deactivation by zeolite pore blockage is prevented, and though it has been reported that by-products (methane and carbon monoxide) are produced by thermal decomposition of DME which is converted when being in contact with a solid acid catalyst of methanol, the problem described above does not particularly occur within the surface acid site range of the present invention due to the nature of the diesel raw material of the present invention, and the acid site of zeolite needs to be controlled to facilitate the decomposition of hydrocarbons such as isoparaffin, naphthene, and aromatics included in a diesel raw material at a high content.

The total acid site and the surface acid site of the phosphorus-supported zeolite may satisfy the following Equation 2:

$$0.2 \leq As / Aa \leq 0.4 \qquad [ Equation 2 ]$$

wherein As is a surface acid site (mmol/g) of the phosphorus-supported zeolite, and Aa is a total acid site (mmol/g) of the phosphorus-supported zeolite.

The effect described above may be further improved by controlling the acid site of a specific zeolite as described in Equation 2 in the catalytic cracking reaction of the mixed raw material of diesel and methanol, and the total and surface acid sites of the present invention may be measured by a wet analysis method using an indicator.

In addition, the catalyst of the present invention may have a residence time of an isobutane gas in the pores of the phosphorus-supported porous zeolite of 0.037 to 1.288 min/g, specifically 0.432 to 1.064 min/g. The catalyst of the present invention has the residence time of the isobutane gas in the designed range, thereby appropriately adjusting a contact time of the reactants and the catalyst to improve the selectivity of light olefins. Specifically, when the contact time of the reactants and the catalyst is excessively increased, a side reaction progresses rapidly to lower the selectivity of light olefins, and on the contrary, when the contact time is too short, a reaction conversion rate is decreased to lower the light olefin yield.

In general, diesel contains isoparaffin, naphthene, aromatics, and the like a lot, and as an example, most of hydrocarbon having branches and naphthene included in the components are cyclohexane having at least two alkyl groups. Considering the characteristics of the raw material, isobutane may be appropriate as a probe molecule formed of a structure similar to the raw material, and on the contrary, propane and the like may not be appropriate as an indicator for determining the pore characteristics of the zeolite catalyst of the present invention.

Meanwhile, the residence time of isobutane in the pores may be measured and calculated by an Autochem II2920 adsorption analyzer available from Micrometritics. In the present invention, the residence time of the isobutane gas may refer to an increased amount of time, which is, as compared with a time taken to detect isobutane in a thermal conductivity detector (TCD) after injecting an isobutane gas (25 ml/min) and He (50 ml/min) simultaneously at flow rates at 70° C. for 3 minutes into the device into which the catalyst is not charged, an increased time taken to detect the isobutane gas in TCD while injecting isobutane and helium in pulse under the same conditions into the device into which the catalyst of the present invention is charged.

The phosphorus-supported zeolite may satisfy the following Equation 3

$$2. \leq At / Aa ( min / m mol ) \leq 4. \qquad [ Equation 3 ]$$

wherein At is the residence time (min/g) of the isobutane gas in the pores of the catalyst, and Aa is the total acid site (mmol/g) of the phosphorus-supported zeolite.

The At/Aa ratio may be, specifically 2.0 min/mmol or more or 3.0 min/mmol or more and 4.0 min/mmol or less, 3.8 min/mmol or less, or 3.6 min/mmol or less, preferably 2.0 to 3.8 min/mmol or 3.0 to 3.6 min/mmol. In order to increase the selectivity of light olefins from methanol and diesel simultaneously in the present invention, phosphorus is supported on ZSM zeolite showing the best resolution to adjust the acid site, and also, an olefin yield may be rapidly increased by designing pores optimized for the specific area of P/Al and a by-product yield may be relatively decreased simultaneously.

Another exemplary embodiment of the present invention provides a method for preparing a catalyst used in a reaction for preparing light olefins by catalytic cracking of a mixture including diesel and methanol. The preparation method is characterized by including: (a) mixing a porous zeolite having pores and a phosphorus compound at contents corresponding to a P/Al mole ratio of 0.1 to 2.5 in a solvent; (b) drying the mixture at 100 to 150° C. for 5 to 20 hours; and (c) firing a product of the drying at 400 to 600° C. for 2 to 10 hours.

The method for preparing a catalyst may be a method for preparing a catalyst used in the reaction for preparing light olefins by catalytic cracking of the mixture including diesel and methanol according to an exemplary embodiment.

(a) is a step of supporting a phosphorus compound by mixing a porous zeolite having pores and the phosphorus compound in a solvent, which is characterized by mixing them in a solvent so that a P/Al mole ratio is 0.1 to 2.5.

The porous zeolite having pores and the P/Al mole ratio are as described above.

The phosphorus compound basically includes phosphorus (P) in a chemical structure and may be a compound capable of reacting the acid site of zeolite. For example, it may include phosphoric acid, ammonium phosphate, and/or alkyl phosphate, and specifically, may include phosphoric acid (H₃PO₄), H₂NH₄PO₄, H(NH₄)₂PO₄, and/or an organophosphorus compound (organic phosphite) compound having a size to penetrate into the zeolite pores. Meanwhile, when NaH₂PO₄, Na₂HPO₄, Na₃PO₄, and the like based on sodium phosphate are used, Na⁺ cations produced during phosphorus support affect the zeolite acid site, so that the characteristics of the solid acid catalyst may not be implemented well, which is thus not preferred.

The solvent may be any solvent used in the art, and for example, distilled water may be used.

As a method for supporting a phosphorus compound inside the pores of the zeolite and/or on the surface of zeolite, generally zeolite is impregnated in a solution containing the phosphorus compound or chemical vapor deposition (CVD) and the like are used. When the chemical vapor deposition is used, the phosphorus compound is heated into gas, and then the phosphorus compound in a gas state is diffused into the pores of zeolite and reacted with the acid site, but this method may not be appropriate for being applied to the present invention.

(b) is a step of drying the compound at 100 to 150° C. for 5 to 20 hours, and the phosphorus compound may be uniformly supported inside the pores of zeolite and/or on the surface of zeolite by removing the solvent. Since the drying step is performed at 100 to 150° C. or 100 to 130° C. for 5 to 20 hours or 7 to 15 hours, the phosphorus compound may be uniformly supported in the zeolite, which makes adjustment of the residence time of an isobutane gas in the pores of the catalyst and control of the acid site in the catalyst easy, in the phosphorus-supported zeolite of the present invention.

The drying (b) may further include evaporating the solvent in the mixture of the mixing (a) in an evaporator at room temperature, and for example, a rotary evaporator may be used for solvent evaporation, but the present invention is not limited thereto.

The firing (c) may be performed at 400 to 600° C., for example, 450 to 550° C. for 2 to 10 hours or 3 to 8 hours, but the firing temperature and the firing time are not limited to the ranges. The amount of the acid site in the catalyst may be decreased and pore blocking is derived by a chemical reaction of aluminum in the zeolite and added phosphorus, which may change pore properties.

Another exemplary embodiment of the present invention provides a method for preparing light olefins by catalytic cracking of reactants including diesel and methanol in the presence of the catalyst of the present invention. Herein, the light olefin may include ethylene, propylene, or both of them. In addition, a diesel/methanol weight ratio in the reactants may be 0.1 to 5. The reaction may be performed in a stationary bed reactor, and may be performed under the reaction conditions of a reaction temperature of 600 to 700° C., a reaction pressure of 0.1 to 2 bar, a weight ratio of catalyst/total reactants of 2 to 20, and an injection flow rate of reactants of being injected at a rate of 0.5 to 3.0 ml/h based on the hydrocarbon injection amount (hydrocarbon rate). The catalytic cracking reaction may be performed so that an olefin weight ratio between the reactants and the catalytic cracking product is 1:5 to 1:100. However, this is an example, and the present invention is not limited thereto.

The present inventors studied various catalytic compositions for producing light olefins by catalytic cracking of a mixed raw material of diesel and methanol, and as a result, invented a catalyst component which has high activity/high durability and may obtain a high light olefin yield, and a method for preparing the same.

Hereinafter, the preferred examples and the comparative examples of the present invention will be described. However, the following examples are only a preferred exemplary embodiment of the present invention, and the present invention is not limited thereto.

### EXAMPLES

### Examples 1 to 5

### Preparation Example 1

### Preparation of Zeolite Used in the Present Invention

ZSM-5 and ZSM-11 were prepared according to the document (Journal of Materials Science, 51 (2016) 3735-3749, Fuel Processing Technology 91 (2010) 449-455), and also, Y, Beta, MOR is available from Zeolyst and SAPO-34 (China holding company) and SAPO-11 are commercial products available from ACS material.

### Preparation Example 2

### Preparation of Phosphorus-Supported ZSM-11 Zeolite

10 g of ZSM-11 (Si/Al=11) zeolite was uniformly mixed with a mixed aqueous solution of 0.3 g of phosphoric acid (H₃PO₄, 85%) and 4.7 g of distilled water, and then phosphoric acid was supported by removing the solvent using a rotary evaporator. The sample prepared above was dried at 110° C. for 12 hours, fired at 500° C. for 5 hours, and cooled to room temperature, and zeolite having P/Al=0.2 of phosphorus (P) to the content of aluminum (Al) in zeolite was prepared and used in the present experiment (Example 1).

The process was performed as described above, with a difference in the mole ratio of zeolite and phosphoric acid, thereby preparing ZSM-11 zeolite having P/Al=0.4, 0.6, 0.8, 1.0 as compared with the content of aluminum (Al) in zeolite.

### EXPERIMENTAL EXAMPLES

### Experimental Example 1

### Preparation of Light Olefins by Catalytic Decomposition Reaction of Diesel and Methanol Mixed Raw Material by Zeolite Type

A stationary bed reactor was filled with 0.1 g of the catalyst prepared in Preparation Example 1 of the present invention, diesel and methanol as the reactants were used at the same weight ratio and injected, the injection flow rate of the reactants was 2.1 ml/h based on the hydrocarbon injection amount (hydrocarbon rate), and the reaction was performed at a reaction temperature of 650° C. and a reaction pressure of 1 bar. The composition of diesel used in the present Experimental Example 1 was as shown in the following Table 1, and the results of the mixed catalytic cracking reaction of diesel and methanol are summarized in the following Table 2.

TABLE 1

| Carbon | n- | iso- | | | | |
|---|---|---|---|---|---|---|
| No. | paraffin | paraffin | Olefin | Naphthene | Aromatics | Total |
| C₆-C₈ | 0.4 | 0.7 | 0.2 | 1.9 | 1.3 | 4.4 |
| C₉-C₁₂ | 3.1 | 9.5 | 2.2 | 25.3 | 7.6 | 47.8 |
| C₁₃-C₁₉ | 7.4 | 14.9 | 2.9 | 15.9 | 2.9 | 41.7 |
| C₁₉-C₃₂ | 2.3 | 1.6 | 0 | 0 | 0 | 6 |
| Sum | 13.2 | 26.7 | 5.2 | 43.1 | 11.7 | 99.9 |

(In Table 1, the unit is wt %.)

TABLE 2

| Zeolite | | | | | | |
|---|---|---|---|---|---|---|
| type | ZSM-11 | ZSM-5 | Y | Beta | SAPO-11 | Mordenite |
| Si/Al | 11 | 11 | 15 | 12.5 | 0.07 | 10 |
| CO | 0.9 | 1.4 | 1.8 | 1.7 | 0.9 | 1.0 |
| CH₄ | 5.7 | 6.5 | 6.9 | 8.5 | 4.6 | 4.4 |
| C₂H₆ | 4.7 | 4.9 | 4.2 | 4.5 | 3.9 | 3.7 |
| C₂H₄ | 14.7 | 14.9 | 10.1 | 9.6 | 9.8 | 9.6 |
| C₃H₈ | 3.1 | 3.5 | 0.7 | 0.9 | 0.7 | 0.7 |
| C₃H₆ | 10.0 | 9.3 | 9.1 | 8.9 | 9.1 | 9.6 |
| C₄+ | 1.7 | 1.5 | 1.9 | 2.0 | 1.8 | 1.9 |
| Gas total | 41.0 | 40.5 | 34.8 | 36.1 | 30.8 | 30.9 |
| C₂H₄ & | 24.7 | 24.2 | 19.2 | 18.5 | 18.9 | 19.2 |
| C₃H₆ | | | | | | |

(In Table 2, the unit is wt %, and Si/Al is a SiO₂/Al₂O₃ mol ratio.)

From the results of Table 2, it was confirmed that ZSM-11 which is a medium pore zeolite had the best olefin selectivity, as zeolite which had the lowest production amount of by-products such as carbon monoxide and methane and was able to selectively produce a lot of olefins by simultaneously decomposing diesel and methanol.

### Experimental Example 2

### Preparation of Light Olefins by Catalytic Decomposition Reaction of Diesel and Methanol Mixed Raw Material Using Phosphorus-Supported ZSM-11 Zeolite

### Comparative Examples 1 to 4

ZSM-11 zeolite having P/Al, the content of phosphorus (P) to the content of aluminum (Al)=0, 1.5, 2.0, 2.5 was prepared in the same manner as in Preparation Example 2, except that the mole ratio of zeolite and phosphoric acid was changed.

A stationary bed reactor was filled with 0.1 g of each of the catalysts prepared in Examples 1 to 5 of the present invention and the catalyst prepared in Comparative Example 1, and the reaction was performed at a reaction temperature of 650° C. and a reaction pressure of 1 bar. At this time, diesel and methanol were used at the same weight ratio as the reactants, and the injection flow rate was 0.87 g/h based on the hydrocarbon rate.

The total acid site, the surface acid site, and the total/surface acid site ratio of the phosphorus-supported zeolite used in the present Experimental Example 2, and the residence time of isobutane are as shown in the following Table 3, and the results of the diesel and methanol mixed catalytic cracking reaction are summarized in the following Table 4.

The total acid sites of the samples prepared from Examples 1 to 5 of the present invention and Comparative Examples 1 to 4 were analyzed by an ammonia heating desorption (NH₃-TPD) method using Hewlett-Packard 5890 series II gas chromatograph equipped with a thermal conductivity detector (TCD), the amount of the used sample was 0.1 g, the amount of ammonia (mmol/g) desorbed by degassing with He (50 ml/min) at 550° C. for 2 hours, adsorbing 10 mol % of NH₃ (30 mL/min) at 150° C. for 30 minutes, and then heating to 150 to 700° C. by 10° C. was measured using TCD, and the amount of the total acid site (mmol/g) was calculated from the amount.

In addition, the amount of the surface acid site was able to be measured by a wet analysis method, and a probe capable of reacting the acid site was allowed to react selectively only with the acid site present on the surface of the catalyst, using basic cyclohexylamine which is larger than a pore opening of zeolite. 0.1 g of the prepared catalyst and 10 g of octane as a dispersant were mixed, and 0.03 g of methyl red as an indicator was added. The indicator was red when the prepared solution was acidic, but changed to yellow by a neutralization reaction when basic cyclohexylamine was added, and the amount of cyclohexylamine which derived the neutralization reaction varied depending on the amount of acid in the solution. That is, as the acid site was more present on the surface of the catalyst, the amount of cyclohexylamine was increased, and the amount of the surface acid site (mmol/g) was calculated from the amount (mmol/g).

In addition, the residence time of isobutane in the pores was measured and calculated by an Autochem II2920 adsorption analyzer available from Micrometritics. Specifically, as compared with a time taken to detect isobutane in a thermal conductivity detector (TCD) after injecting an isobutane gas (25 ml/min) and He (50 ml/min) simultaneously at flow rates at 70° C. for 3 minutes into the device into which the catalyst was not charged, an increased amount of time taken to detect the isobutane gas in TCD while injecting isobutane and helium in pulse under the same conditions into the device into which the catalyst of the present invention was charged was measured as the residence time of isobutane in pores.

TABLE 3

| | P/Al | Total acid site (Aa) | iso-butane residence |
|---|---|---|---|
| | (mol ratio) | (mmol/g) | time (min/g) |
| Comparative | 0.0 | 0.621 | 1.288 |
| Example 1 | | | |
| Example 1 | 0.2 | 0.464 | 1.200 |
| Example 2 | 0.4 | 0.384 | 1.160 |
| Example 3 | 0.6 | 0.320 | 1.064 |
| Example 4 | 0.8 | 0.239 | 0.856 |
| Example 5 | 1.0 | 0.208 | 0.432 |
| Comparative | 1.5 | 0.192 | 0.212 |
| Example 2 | | | |
| Comparative | 2.0 | 0.179 | 0.178 |
| Example 3 | | | |
| Comparative | 2.5 | 0.161 | 0.109 |
| Example 4 | | | |

TABLE 4

| | Comparative | | | | | |
|---|---|---|---|---|---|---|
| | Example 1 | Example 1 | Example 2 | Example 3 | Example 4 | Example 5 |
| P/Al | 0.0 | 0.2 | 0.4 | 0.6 | 0.8 | 1.0 |
| (mol ratio) | | | | | | |

Yield (wt %)

| | | | | | | |
|---|---|---|---|---|---|---|
| CO | 0.9 | 0.9 | 0.8 | 0.8 | 0.8 | 0.7 |
| CH₄ | 5.7 | 5.3 | 5.0 | 4.8 | 4.2 | 4.1 |
| C₂H₆ | 4.7 | 4.6 | 4.5 | 4.3 | 4.0 | 3.8 |
| C₂H₄ | 14.7 | 15.7 | 17.6 | 17.5 | 19.0 | 16.4 |
| C₃H₈ | 3.1 | 3.5 | 3.0 | 2.3 | 1.8 | 1.0 |
| C₃H₆ | 10.0 | 11.5 | 13.1 | 16.1 | 17.0 | 18.6 |
| C₄+ | 1.7 | 2.2 | 2.1 | 2.7 | 2.5 | 3.4 |
| Gas total | 41.0 | 43.8 | 46.1 | 48.4 | 49.3 | 48.0 |
| C₂H₄ & C₃H₆ | 24.7 | 27.2 | 30.7 | 33.6 | 36.0 | 35.0 |

In general, when phosphorus is supported on zeolite, amorphous aluminum phosphate (AlOP) or partially crystallized AlOP are formed by a chemical reaction of aluminum and added phosphorus to form an acid site in zeolite to decrease the amount of the acid site, and a pore blocking phenomenon is derived by the changed structure to change the pore properties. In the present invention, in order to increase the selectivity of light olefins from methanol and diesel simultaneously, phosphorus was supported on ZSM-11 zeolite showing the best resolution to adjust the acid site, and as a result of the experiment, it was confirmed that the olefin yield was rapidly increased in a specific area of P/Al and the yield of by-product was relatively decreased.

### Experimental Example 3

### Confirmation of Heat Neutralization Phenomenon During Catalytic Decomposition Reaction Depending on Diesel and Methanol Mixed Raw Material Ratio Using Phosphorus-Supported ZSM-11 Zeolite

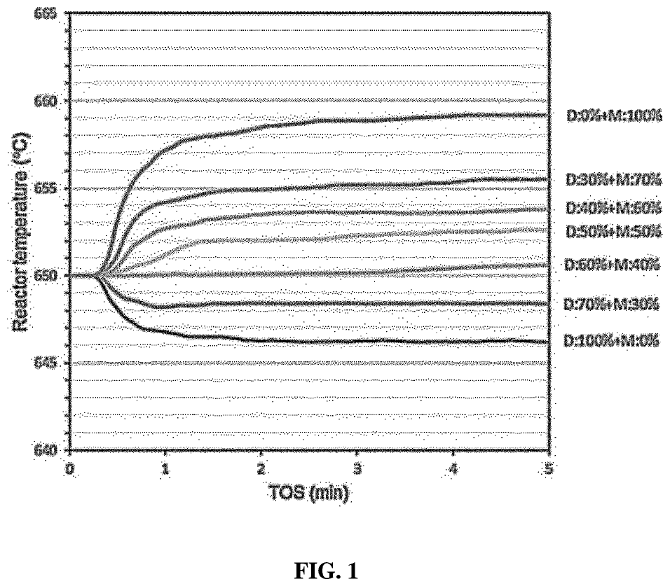

In order to confirm a heat neutralization phenomenon by a mixed decomposition reaction of a diesel decomposition reaction having endothermic reaction characteristics and a methanol conversion reaction having exothermic reaction characteristics according to the present invention, a catalytic decomposition reaction on the catalyst of Example 1 was performed under the same conditions as in Experimental Examples 1 and 2, the mixed decomposition reaction was performed with the mixed ratio of methanol to diesel being changed from 0 wt % to 100 wt %, temperature changes of the catalytic layer in the reactor were observed, and the results are shown in FIG. 1.

As shown in FIG. 1, when a ratio of the mixed weight of methanol to diesel was 50%, it was confirmed that heat neutralization having energy usage of 0 occurred. Thus, it was found that simultaneous decomposition of diesel and methanol may occur in the state of allowing the heat neutralization according to the present invention.

1. A catalyst used in a reaction for preparing light olefins by catalytic cracking of a mixture including diesel and methanol, comprising:
a porous zeolite having pores; and
phosphorus supported inside the pores of the zeolite and/or on a surface of the zeolite,
wherein the catalyst has a residence time of an isobutane gas in the pores of 0.400 to 1.240 min/g.

2. The catalyst used in a reaction for preparing light olefins by catalytic cracking of a mixture including diesel and methanol of claim 1, wherein the catalyst has the residence time of the isobutane gas in the pores of 0.432 to 1.064 min/g.

3. The catalyst used in a reaction for preparing light olefins by catalytic cracking of a mixture including diesel and methanol of claim 1, wherein the catalyst satisfies the following Equation 1:
$$0.1 \leq P / A 1 \leq 2.5 \qquad [ Equation 1 ]$$
wherein P is a content (mol) of phosphorus supported on the zeolite, and Al is a content (mol) of aluminum in the zeolite.

4. The catalyst used in a reaction for preparing light olefins by catalytic cracking of a mixture including diesel and methanol of claim 3, wherein the P/Al mole ratio is 0.4 to 0.9.

5. The catalyst used in a reaction for preparing light olefins by catalytic cracking of a mixture including diesel and methanol of claim 1, wherein the phosphorus-supported zeolite has a total acid site (Aa) of 0.200 to 0.620 mmol/g.

6. The catalyst used in a reaction for preparing light olefins by catalytic cracking of a mixture including diesel and methanol of claim 5, wherein the phosphorus-supported zeolite has a surface acid site (As) of 0.050 to 0.150 mmol/g.

7. The catalyst used in a reaction for preparing light olefins by catalytic cracking of a mixture including diesel and methanol of claim 1, wherein the phosphorus-supported zeolite satisfies the following Equation 2:
$$0.2 \leq As / Aa \leq 0.4 \qquad [ Equation 2 ]$$
wherein As is a surface acid site (mmol/g) of the phosphorus-supported zeolite, and Aa is a total acid site (mmol/g) of the phosphorus-supported zeolite.

8. The catalyst used in a reaction for preparing light olefins by catalytic cracking of a mixture including diesel and methanol of claim 1,
wherein the zeolite has the Si/Al mole ratio of 200 or less and includes at least one selected from the group consisting of ZSM-11, ZSM-5, Beta, Mordenite, chabazite, or Ferrierite.

9. The catalyst used in a reaction for preparing light olefins by catalytic cracking of a mixture including diesel and methanol of claim 8, wherein the zeolite has the Si/Al mole ratio of 8 to 30 and is ZSM-11 or ZSM-5.

10. The catalyst used in a reaction for preparing light olefins by catalytic cracking of a mixture including diesel and methanol of claim 1, wherein the phosphorus-supported zeolite satisfies the following Equation 3:
$$2. \leq At / Aa ( min / m mol ) \leq 4. \qquad [ Equation 3 ]$$
wherein At is the residence time (min/g) of the isobutane gas in the pores of the catalyst, and Aa is the total acid site (mmol/g) of the phosphorus-supported zeolite.

11. A method for preparing the catalyst of claim 1, the method comprising:
(a) mixing a porous zeolite having pores and a phosphorus compound at contents corresponding to a P/Al mole ratio of 0.1 to 2.5 in a solvent;
(b) drying the mixture at 100 to 150° C. for 5 to 20 hours; and
(c) firing a product of the drying at 400 to 600° C. for 2 to 10 hours.

12. The method for preparing the catalyst of claim 11, wherein the phosphorus compound includes phosphoric acid, ammonium phosphate, and/or alkyl phosphate.

13. The method for preparing the catalyst of claim 11, wherein the porous zeolite having pores has a Si/Al mole ratio of 200 or less and includes at least one selected from the group consisting of ZSM-11, ZSM-5, Beta, Mordenite, chabazite, or Ferrierite.

14. The method for preparing the catalyst of claim 11, wherein the drying (b) further includes evaporating the solvent in the mixture of the mixing (a) in an evaporator at room temperature.

15. A method for preparing light olefins by catalytic cracking of reactants including diesel, methanol, or a mixed raw material thereof in the presence of the catalyst of claim 1.

16. The method for preparing light olefins of claim 15, wherein the mixed raw material has a weight ratio of diesel to methanol of 0.1 to 5.

17. The method for preparing light olefins of claim 15, wherein the catalytic cracking is performed under reaction conditions of a reaction temperature of 600 to 700° C., a reaction pressure of 0.1 to 2 bar, and a weight ratio of the catalyst to the reactants of 2 to 20.

18. The method for preparing light olefins of claim 15, wherein the reactants and a product of the catalytic cracking has an olefin weight ratio of 1:5 to 1:100.
